(19) United States
(12) Patent Application Publication
(10) Pub. No.: US 2017/0259449 A1
(43) Pub. Date: Sep. 14, 2017

(54) Silicone Membrane Slitting Machine and Method

(71) Applicant: E. Aubrey Woodroof, Carlsbad, CA (US)

(72) Inventor: E. Aubrey Woodroof, Carlsbad, CA (US)

(21) Appl. No.: 15/416,560

(22) Filed: Jan. 26, 2017

Related U.S. Application Data
(63) Continuation-in-part of application No. 14/524,752, filed on Oct. 27, 2014, now abandoned.

(51) Int. Cl. B26F 1/20; A61F 13/00; B26D 1/62

(52) U.S. Cl. CPC B26F 1/20; B26D 1/626; A61F 13/00995

(57) ABSTRACT

A mechanism and a method for slitting silicone membranes using automated slitting blades is presented. The pattern of slits in the membrane can be adjusted by using a cutting cylinder comprised of a plurality of circular cutting gears assembled in parallel.

The cutting cylinder is of indefinite length, but in the preferred embodiment is approximately 15″ long and 3″ in diameter. The silicone membrane can range in thickness from 0.005″ to 0.01″ inch.

### RELATED APPLICATIONS

This application is a Continuation-in-Part of U.S. patent application Ser No. 14/524,752, filed Oct. 27, 2014, currently copending, which was a Continuation-in-Part of U.S. patent application Ser. No. 14/159,332 filed Jan. 20, 2014, which was a Continuation-in-Part of U.S. patent application Ser. No. 13/897,430, filed May 19, 2013.

### FIELD OF THE INVENTION

This invention relates to the manufacture of dressings and bandages for chronic wounds, including hernias, and of dermatological masks and coverings.

### BACKGROUND OF THE INVENTION

Wound management involves removal of all non-viable tissue at the wound site, preserving the remaining viable tissue, and providing a moist but not wet environment. An example of successful burn wound dressing is Biobrane, granted U.S. Pat. No. 4,725,279. In 1979 Biobrane was initially studied by American Burn Surgeons; it is still popular world-wide.

In 2007 new art was introduced by this inventor with AWBAT and then with AWBAT Plus, granted U.S. Pat. No. 7,815,931 and covered by several copending patent applications. The key to the success of these products was better porosity in the dressing.

Recently, this inventor has revisited the art of dressing design. The present invention allows passage of fluid adjacent to the wound through the primary dressing into a secondary absorbent dressing as well as improving the kinetics of uninterrupted wound healing. Technology of this dressing has evolved into a new product which possesses all the characteristics and attributes known to be important for optimal wound healing, as well as containing certain advances that result in minimization of wound desiccation and infection complication.

The methods described above for designing and providing wound dressings are also applicable to the process of treating skin for medicinal or cosmetic reasons. The present invention addresses these areas as well,

### SUMMARY OF THE INVENTION

Wound sites have variable amounts of exudate/transudate/plasma present, from dry to weepy. The clinician must cleanly debride the wound, close it and manage wound healing in a moist but not wet environment to achieve optimal results in both acute and chronic wounds.

The present invention provides a dressing that possesses all the properties and attributes of an ideal skin substitute and, in addition, has ‘variable porosity’ controlled by the clinician from zero porosity to what the wound requires. The present invention enables the clinician to move the fluid exuding from the wound through the primary dressing into an absorbent secondary dressing without disturbing the kinetics of healing or causing pain to the patient.

The present invention is cost effective at every level. Patients get their wounds managed with minimal pain and optimal healing times. The dressing is cost effective as the hospital needs to inventory only one primary dressing for acute wounds (burns) and one for chronic wounds; each has a two year shelf-life at room temperature.

The present invention is composed of two biological layers sprayed on in separate operations. The first layer sprayed onto the nylon side of the “variable porosity” silicone membrane will be: (1) a solution of pure Aloe (Aloesin, Immuno10, Qmatrix and Loesyn—each hydrophilic and hygroscopic.); (2) a solution of pure Aloe and hypoallergenic USP Pharmaceutical Grade porcine gelatin; or (3) a fine suspension of pure Aloe, gelatin and Extra Cellular Matrix (ECM—as fine insoluble particles or hollow spheres in water—the latter possesses improved healing properties). In vitro, the Aloe component has been demonstrated to cause a variety of cells to attach and proliferate; as well as increase synthesis of collagen and alpha smooth muscle actin. ECM may be added to the biologicals described above and is a mixture from human fibroblasts that is known to cause rapid cell proliferation and tissue growth.

Previous wound dressings and skin substitutes, as taught in U.S. Pat. No. 7,815,931 contain gelatin, a pure Aloe component, chondroitin 4 & 6 sulfate, and vitamin C & E. In contrast the current dressing will have two layers of biologicals applied in separate spraying operations as described above. The first coat will contact the wound after the second coat of hypoallergenic bovine spongiform encephalopathy (BSE)—free United States Pharmaceutical (USP)—grade gelatin interacts with fibrin in the wound to achieve early adherence, The second coat of biologicals stimulates the healing process during the interval where the dressing invention is in contact with the wound and is stable requiring 100 degree water for 30 minutes to remove from the “variable porosity” silicone/nylon surface.

An alternate embodiment of the invention is a slitted silicone membrane (SSM) which resembles the main embodiment in that it comprises a slitted silicone sheet approximately 0.005 inches thick with no nylon backing. The sheet is slitted in the same manner as the slitted silicon nylon matrix above.

The present invention is a mechanical means by which the above-described slitted silicon nylon matrix (SSNM) and the SSM are processed from unslitted, continuous materials into slitted dermatological and surgical dressings.

### BRIEF DESCRIPTION OF THE FIGURES

FIG. 1. Perspective view of the slitting machine assembled

FIG. 2. Exploded view of the slitting machine

FIG. 3. Cross-Section of the assembled slitting machine

FIG. 4. Perspective view of one horizontal slitting gear

FIG. 5. Perspective view of one vertical slitting gear

FIG. 6. Sample pattern for vertically-slitted silicone membrane

FIG. 7. Sample pattern for vertically- and horizontally-slitted silicone membrane

### DETAILED DESCRIPTION

The SSNM and SSM used by the present invention are similar in composition to earlier skin substitutes in that they each have as a minimum a thin silicone component and optionally an underlying thin knitted nylon component. The present invention possesses “variable porosity” controlled by the clinician; the pore size in the thin, slitted silicone will be essentially zero with no stretch applied to the membrane, in relaxed mode, to a higher porosity proportional to the stretch applied. The present invention possesses a composition of biological coatings applied to both the silicone component and the optional nylon component.

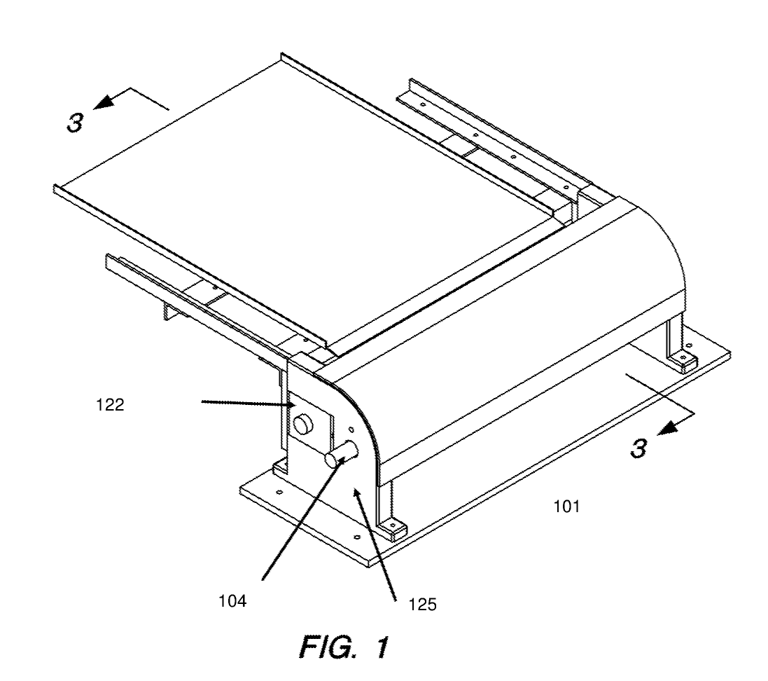

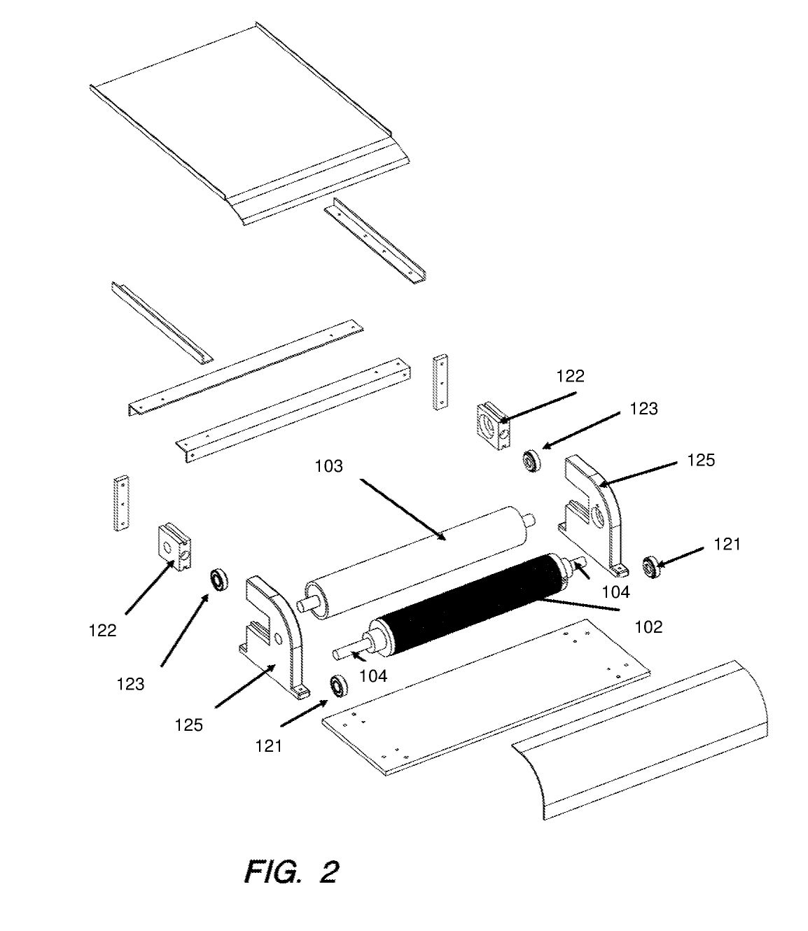

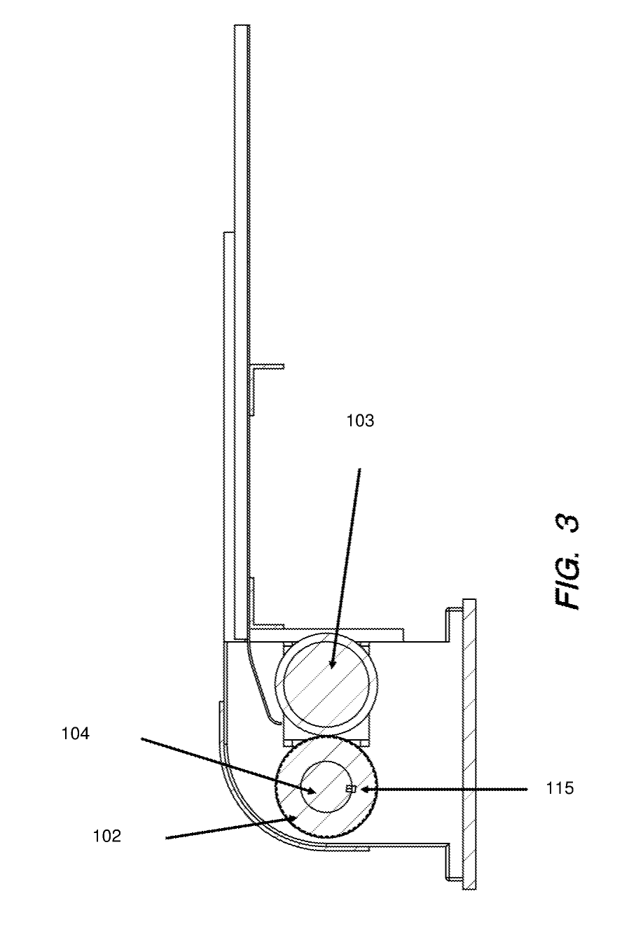

The slitting machine 101 is shown in perspective view in FIG. 1. There is an exploded view of its components in FIG. 2, and a cross-section view in FIG. 3.

The slitting machine 101 is comprised primarily of two rollers, the slitting roller 102 and the backing roller 103. When the slitting roller 102 is pressed against the backing roller 103 with an SSM or SSNM membrane between them, it cuts a pattern of slits through the membrane as the slitting roller turning means (not shown) turns the axle 104 of the slitting roller 102.

This is the fundamental process of the invention, using the blades of the slitting roller 102 to make slits in a length of SSNM or SSM continuously.

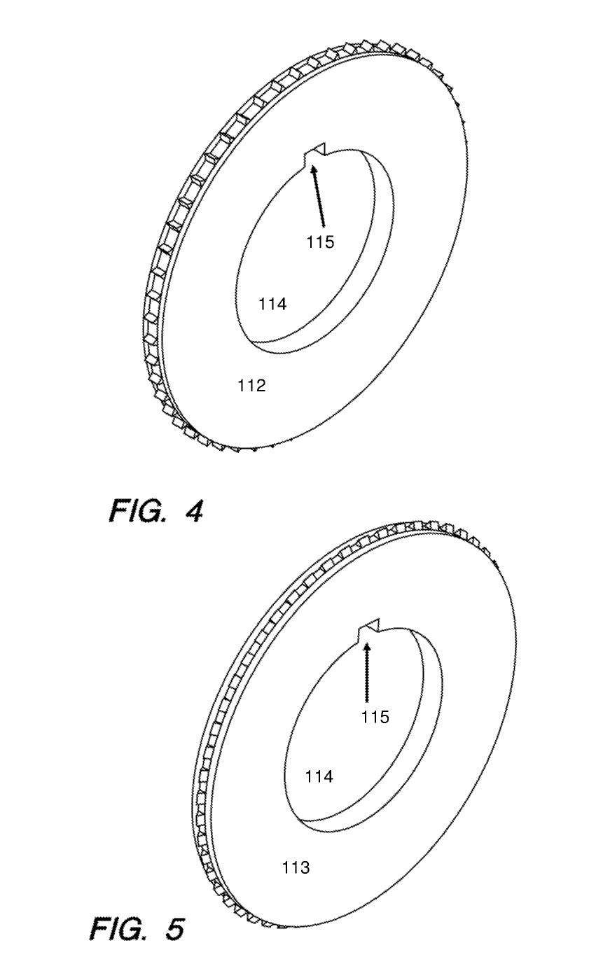

The slit openings are made by the slitting machine 101 after the silicone component has been cured. A perspective view of two of the component cutting gears 112, 113 that can be assembled into the slitting machine 101 slitting roller 102 are shown in FIG. 4 and FIG. 5.

The cutting gears 112,113 are flat cylinders each with an axis hole 114 cut through their center and a registration notch 115 that is used to assure that the individual gears are oriented correctly to each other when assembled side-by-side into the slitting roller 102. FIG. 4 shows a cutting gear 112 with blades in a horizontal orientation and FIG. 5 shows a cutting gear 113 with cutting blades in a vertical orientation.

As in FIG. 2, the slitting roller 102 and the backing roller 103 are independent of each other and mounted in the axle rest 125 as shown. In the cross-section diagram, FIG. 3, the slitting roller 102 and the backing roller 103 are shown assembled and pressing against each other.

SSNM or SSM material is passed between the rollers 102,103 in flat sheets of varying widths. These sheets are between 0.01 and 0.005 inches in thickness. In the case of slitting of the SSNM, the silicone layer is presented to the blades of the slitting roller 102 before the slitting action is performed.

The means used to turn the slitting roller 102 and thereby to operate the invention can be a hand crank or some motor-driven mechanism. The preferred embodiment of the invention uses a hand crank attached to the slitting roller axle 104. The action of turning the slitting roller 102 draws the SSNM or SSM material under the blades of the slitting roller 102.

The slitting machine 101 holds the two rollers 102,103 in proximity to each other in the axle rest 125. The slitting roller axle 104 is held in the axle rest 125 by two slitting roller nuts 121. The backing roller 103 is held adjustably in place in the axle rest 125 by the backing roller adjustment device 122 and secured by the backing roller nuts 123. By manipulating the backing roller adjustment device 122, the backing roller 103 can be positioned against the slitting roller 102 at the desired distance.

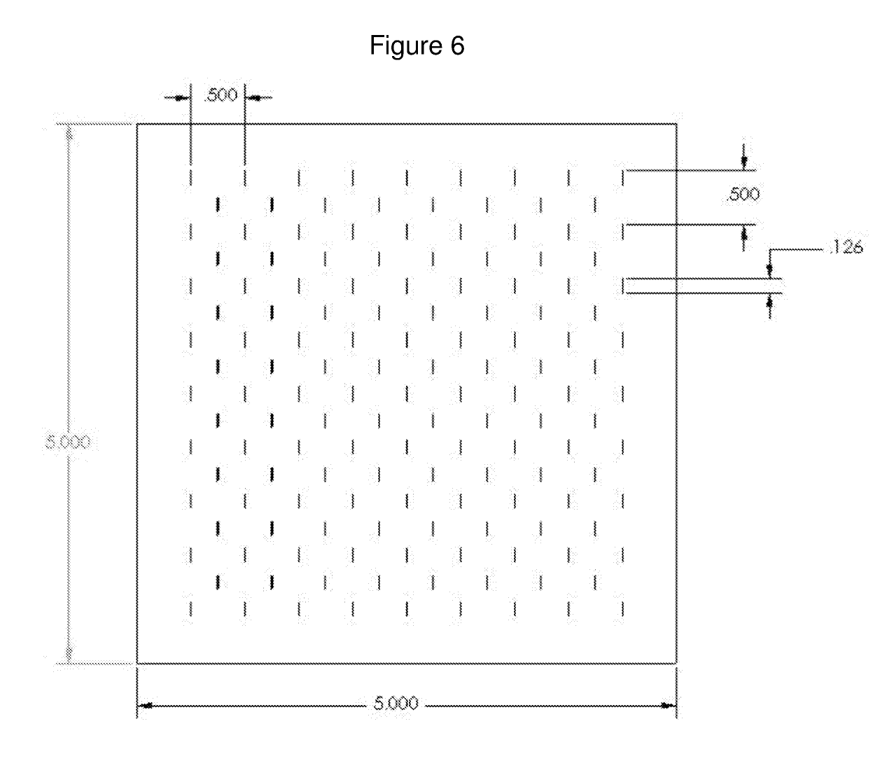

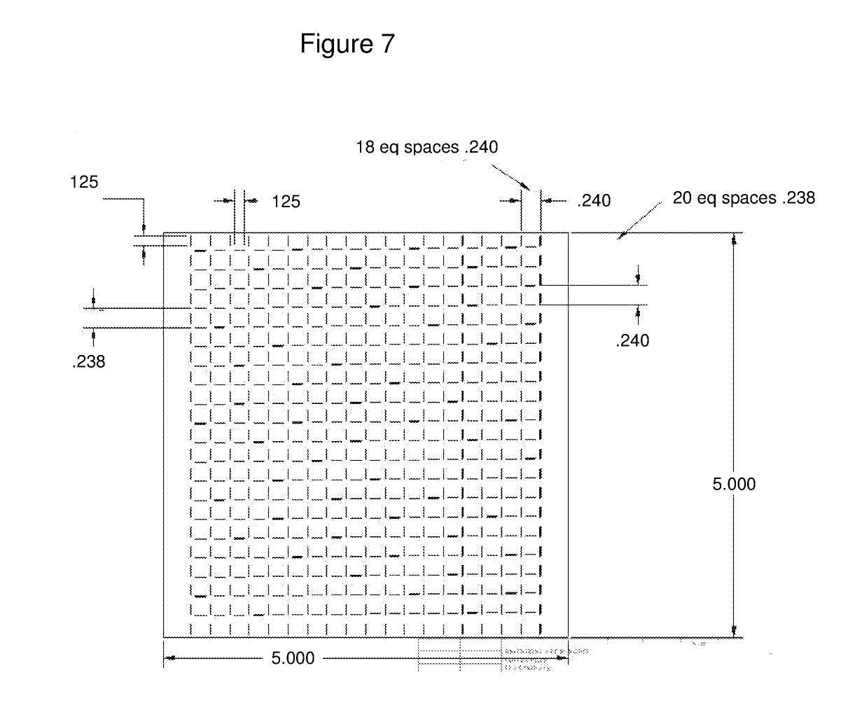

The preferred embodiment of the SSNM and SSM after slitting is shown in examples contained in FIG. 6 and FIG. 7. Slits can be made in an alternating orientation, with vertical and horizontal slits, as in FIG. 7, or in a parallel vertical orientation as in FIG. 6.

The slits made in the silicone are approximately 0.125′ long with a space of 0.50′, between the slits; off-set parallel rows of slits are 0.25″ apart. Rows of slits perpendicular to the above are also 0.125″ long with a space of 0.50″ between the next slit; off-set parallel rows of slits are 0.25″ apart.

Slitted in the alternating orientation configuration, the silicone/nylon membrane can be stretched in any direction and the slits will open. Porosity therefore increases proportionally to the amount of stretch applied in either direction. Obviously, there is a maximum amount of stretching of the preferred embodiment before the dressing fails.

In the SSNM, the knitted nylon component would be the thinnest commercially-available mesh, the preferred material nylon. Either 12/1 or 15/1 denier filament would be used to knit the mesh. Alternatively, polypropylene or other filaments used in mesh hernia repair devices could be used. The mesh would be combined with the medical grade silicone as above to produce the structure of the invention.

The general dimensions of the slitting roller 102 in its preferred embodiment is as follows: the width of each gear 112,113 is 0.180 inches. There are a plurality of gears 112,113 aligned along the slitting roller. In the preferred embodiment, there are 84 gears in the slitting roller 102.

For the vertical (V) and horizontal configuration, the width of each gear (contains multiple blades either V or H) is 0.180″ and the distance between the center of each blade is 0.180″. There are 84 gears aligned along the cutting cylinder. Each gear has 53 cutting blades. The cutting cylinder is 15.12 inches long. The diameter (D) of the cutting cylinder is 3.0 inches. The circumference (C=Pi×D) of the cutting cylinder is 9.43 inches.

In a single full rotation of the V+H cutting cylinder cuts an area of 142.56 sq. inches. In this area there are 4,452 slits, each slit is 0.088 inch wide and there is 0.180 inches between the centers of each slit. The number of slits made per square foot of area is 4,464 slits or 31 slits per sq. inch.

In the V only configuration, the width of each V slit is 0.250″. There are 61 gears on the roller with 37 V blades each. Each gear is rotated 90 degrees from the adjacent gear creating an alternating cutting pattern both along the cutting cylinder as well as around the cutting cylinder.

A single full rotation of the cutting cylinder cuts an area of 142.56 sq. inches. In this area, there are 2,257 slits, each slit is 0.250″ wide and there is 0.250″ between the centers of each slit. The number of slits made per square foot of area is 2,280 slits or 16 slits per sq. inch.

As presented, the invention 101 will handle SSNM or SSM material in 15 inch widths and of indefinite length by moving it through the invention 101 at a speed that minimizes the possibility of the material splitting or breaking.

These are the preferred embodiments of the invention. The technology to create this invention is listed in the preferred embodiments of this invention, but other methods are possible and are within the contemplation of this patent.

What is claimed is:

1. A slitting machine, the slitting machine comprised of a slitting roller, a backing roller, and a slitting machine frame,
the backing roller a cylindrical structure comprised of a firm material, the cylindrical structure possessing a backing roller axle running through the length of the backing roller,
the slitting roller comprised of a plurality of cutting gears, each cutting gear is a flat cylinder with an axial hole, each cutting gear possessing a plurality of cutting blades positioned equidistant along the circumference of the cutting gear, each axial hole possessing a circular rim, each cutting gear possessing a registration notch on the rim of the axial hole,
said cutting gears of two types, horizontal cutting gears and vertical cutting gears, the vertical cutting gear possessing some of the plurality of cutting blades where the direction of the cutting blades is in parallel with the circumference of the vertical cutting gear, the horizontal cutting gears possessing some other of the plurality of cutting blades where the direction of the blade is orthogonal to the circumference of the horizontal cutting gear direction of the plurality of blades,
said slitting roller possessing a slitting roller axle, said backing roller and said slitting roller are of identical length and width,
said slitting machine frame comprised of at least an axle rest, a slitting roller nut, a backing roller adjustment device, and a backing roller nut.

2. A method of using the slitting machine of claim 1, comprised of the steps of
A) assembling the slitting roller by
1) selecting the pattern of cuts that are to be produced in the slitted silicon nylon matrix (SSNM) or slitted silicon matrix (SSM) material, placing the appropriate horizontal or vertical cutting gears side-by-side on the slitting roller axle,
2) placing the slitting roller into the axle rest and securing the slitting roller with the slitting roller nuts, then
B) placing the backing roller into the axle rest by means of the backing roller adjustment device and securing the backing roller with the backing roller nuts, then
C) adjusting the backing roller position in the axle rest to the desired distance from the slitting roller by means of the backing roller adjustment device, then
D) presenting SSNM or SSM material to the gap between the slitting roller and the backing roller and turning the slitting roller axle with a slitting roller turning means, then
E) collecting the slitted SSNM or SSM material and setting it aside, then
F) repeating steps D) and E) until the desired amount of slitted material has been produced, then
G) cleaning the cutting blades on the cutting gears of the slitting roller with a solution of 70% isopropyl alcohol and water with a microfiber cloth.
